SEQUENCE LISTING (44 sequences)

SEQ ID NO: 1 - DNA, 45 bp, Artificial Sequence
  Synthetic oligonucleotide
ctggtggcgtaggcaagagtgccttgacgatacagctaattcaga

SEQ ID NO: 2 - DNA, 19 bp, Artificial Sequence
  Synthetic oligonucleotide
atggtcatagctgtttcct

SEQ ID NO: 3 - DNA, 11 bp, Artificial Sequence
  Synthetic oligonucleotide
cgccaacagct

SEQ ID NO: 4 - DNA, 11 bp, Artificial Sequence
  Synthetic oligonucleotide
cgccaccagct

SEQ ID NO: 5 - DNA, 49 bp, Artificial Sequence
  Synthetic oligonucleotide
gctgaaaatgactgaatataaacttgtggtagttggagctggtggcgta

SEQ ID NO: 6 - DNA, 18 bp, Artificial Sequence
  Synthetic oligonucleotide
tgtaaaacgacggccagt

SEQ ID NO: 7 - DNA, 11 bp, Artificial Sequence
  Synthetic oligonucleotide
agctgttggcg

SEQ ID NO: 8 - DNA, 11 bp, Artificial Sequence
  Synthetic oligonucleotide
agctggtggcg

SEQ ID NO: 9 - DNA, 23 bp, Artificial Sequence
  Synthetic oligonucleotide
ttaaccttatgtgtgacatgttc

SEQ ID NO: 10 - DNA, 19 bp, Artificial Sequence
  Synthetic oligonucleotide
tcctgcaccagtaatatgc

SEQ ID NO: 11 - DNA, 21 bp, Artificial Sequence
  Synthetic oligonucleotide
gtgtgacatgttctaatatag

SEQ ID NO: 12 - DNA, 17 bp, Artificial Sequence
  Synthetic oligonucleotide
ctgaattagctgtatcg

SEQ ID NO: 13 - DNA, 49 bp, Artificial Sequence
  Synthetic oligonucleotide
gctgtatcgtcaaggcactcttgcctacaccaccagctccaactaccac

SEQ ID NO: 14 - DNA, 278 bp, Artificial Sequence
  Synthetic oligonucleotide
gtttgtattaaaaggtactggtggagtatttgatagtgtattaaccttatgtgtgacatgttctaatatagtcacattttcattatttttattataagcctgctgaaaatgactgaatataaacttgtggtagttggagctggtggtgtaggcaagagtgccttgacgatacagctaattcagaatcattttgtggacgaatatgatccaacaatagaggtaaatcttgttttaatatgcatattactggtgcaggaccattctttgatacagataaa

SEQ ID NO: 15 - DNA, 50 bp, Artificial Sequence
  Synthetic oligonucleotide
gaaaatgactgaatataaacttgtggtagttggagctggtggcgtaggca

SEQ ID NO: 16 - DNA, 24 bp, Artificial Sequence
  Synthetic oligonucleotide
ttattataaggcctgctgaaaatg

SEQ ID NO: 17 - DNA, 50 bp, Artificial Sequence
  Synthetic oligonucleotide
ttctgaattagctgtatcgtcaaggcactcttgcctacgccaccagctcc

SEQ ID NO: 18 - DNA, 23 bp, Artificial Sequence
  Synthetic oligonucleotide
tattcgtccacaaaatgattctg

SEQ ID NO: 19 - DNA, 11 bp, Artificial Sequence
  Synthetic oligonucleotide
gccacsagctc

SEQ ID NO: 20 - DNA, 11 bp, Artificial Sequence
  Synthetic oligonucleotide
gagctsgtggc

SEQ ID NO: 21 - DNA, 45 bp, Artificial Sequence
  Synthetic oligonucleotide
cctctgactgtaaagttttaagttttatgcgattaccgggctctg

SEQ ID NO: 22 - DNA, 27 bp, Artificial Sequence
  Synthetic oligonucleotide
tgttgttaagaagaggaattgaacctc

SEQ ID NO: 23 - DNA, 11 bp, Artificial Sequence
  Synthetic oligonucleotide
ggcagggcccg

SEQ ID NO: 24 - DNA, 11 bp, Artificial Sequence
  Synthetic oligonucleotide
ggcagagcccg

SEQ ID NO: 25 - DNA, 46 bp, Artificial Sequence
  Synthetic oligonucleotide
tttttgcagatggctttgtggcggcctagtgacaataccgtatatc

SEQ ID NO: 26 - DNA, 46 bp, Artificial Sequence
  Synthetic oligonucleotide
tttttgcagatggctttgtggcggcctagtgacagtacggtatatc

SEQ ID NO: 27 - DNA, 26 bp, Artificial Sequence
  Synthetic oligonucleotide
cgatcgtaaacgtgttccctattttt

SEQ ID NO: 28 - DNA, 44 bp, Artificial Sequence
  Synthetic oligonucleotide
tcgaggtcgacggtatcgatcgtaaacgttttccctattttttt

SEQ ID NO: 29 - DNA, 41 bp, Artificial Sequence
  Synthetic oligonucleotide
ccagtacggactcgtgttagtaccctaaataccctatattg

SEQ ID NO: 30 - DNA, 32 bp, Human papillomavirus type 18
gctggcagctctagattattaactgttggtaa

SEQ ID NO: 31 - DNA, 32 bp, Human papillomavirus type 45
gcaggcagttcccgattattaactgtaggcaa

SEQ ID NO: 32 - DNA, 50 bp, Artificial Sequence
  Synthetic oligonucleotide
tttttgcagatggcgacgtggcggcctagtgaaaataaggtgtatctacc

SEQ ID NO: 33 - DNA, 50 bp, Artificial Sequence
  Synthetic oligonucleotide
tttttgcagatggcttactggcggcctagtgacagtacggtttatctgcc

SEQ ID NO: 34 - DNA, 26 bp, Artificial Sequence
  Synthetic oligonucleotide
cgatcgtaaacgtgttccctattttt

SEQ ID NO: 35 - DNA, 36 bp, Human papillomavirus type 97
tgtctcgcacaagcatattttatcatgctggcagtt

SEQ ID NO: 36 - DNA, 36 bp, Human papillomavirus type 56
taaaacgcactagtatattttatcatgcaggcagtt

SEQ ID NO: 37 - DNA, 50 bp, Artificial Sequence
  Synthetic oligonucleotide
atgctcagacacaattagcgcgacccttagatccagacaactgttcaaac

SEQ ID NO: 38 - DNA, 22 bp, Artificial Sequence
  Synthetic oligonucleotide
tcctttacttactacacctcag

SEQ ID NO: 39 - DNA, 50 bp, Artificial Sequence
  Synthetic oligonucleotide
aactgttcaaactgatgggacccactccatcgagatttcactgtagctag

SEQ ID NO: 40 - DNA, 20 bp, Artificial Sequence
  Synthetic oligonucleotide
gggactcgagtgatgattgg

SEQ ID NO: 41 - DNA, 23 bp, Artificial Sequence
  Synthetic oligonucleotide
ccacattacatacttaccatgcc

SEQ ID NO: 42 - DNA, 50 bp, Artificial Sequence
  Synthetic oligonucleotide
accatgccactttcccttgtagactgttccaaatgatccagatccaattc

SEQ ID NO: 43 - DNA, 12 bp, Artificial Sequence
  Synthetic oligonucleotide
ctacagwgaaat

SEQ ID NO: 44 - DNA, 10 bp, Artificial Sequence
  Synthetic oligonucleotide
tttgsaacag

(19) United States
(12) Patent Application Publication
(10) Pub. No.: US 2012/0225421 A1
(43) Pub. Date: Sep. 6, 2012

(54) KIT AND METHOD FOR SEQUENCING A TARGET DNA IN A MIXED POPULATION

(75) Inventors: Katherine Richardson, Omaha, NE (US); Benjamin Legendre, JR., Omaha, NE (US); Yanggu Shi, Gaithersburg, MD (US)

(73) Assignee: Transgenomic, Inc., Omaha, NE (US)

(21) Appl. No.: 13/407,274

(22) Filed: Feb. 28, 2012

Related U.S. Application Data
(60) Provisional application No. 61/447,490, filed on Feb. 28, 2011.
(60) Provisional application No. 61/532,887, filed on Sep. 9, 2011.

(51) Int. Cl. C12Q 1/68; G01N 21/00; C12Q 1/70

(52) U.S. Cl. 435/5; 435/6.11

(57) ABSTRACT

Methods and kits for sequencing a target DNA sequence in a sample having a related reference sequence are provided herein. In particular, kits and methods for sequencing cancer and cancer therapy associated mutations are described. Also provided are kits and methods for detecting mitochondrial mutations and for differentiating between closely related viral strains.

### CROSS-REFERENCE TO RELATED APPLICATIONS

This patent application claims the benefit of priority of U.S. Provisional Patent Application No. 61/447,490, filed Feb. 28, 2011, and U.S. Provisional Patent Application No. 61/532,887, filed Sep. 9, 2011, both of which are incorporated herein by reference in their entireties.

### INTRODUCTION

The invention pertains to improvements in DNA sequencing target DNA sequences in nucleic acid samples containing other reference sequences. The reference and target sequences may be closely related, e.g. the target sequence may be an allele of the reference sequence, a mutated form of the reference sequence, or a reference sequence from a separate strain or species. In particular, the invention relates to use of a blocking nucleic acid during a DNA sequencing reaction to block sequencing of the reference sequence, but not of the target sequence.

DNA sequencing allows for identification of a specific DNA sequence by using a sequencing primer specific for a particular region of a nucleic acid. The method is very powerful and rapidly provides sequence information as long as the sequencing primer is specific for only one sequence in the sample. A commonly encountered problem in sequencing is when the population of sequences is mixed, such that the sequencing primer allows for two sequences that cannot be properly resolved. The need to identify and sequence a target sequence in a background of related reference sequences persists with newly developed sequencing methods.

### SUMMARY

Kits and methods for sequencing a target DNA sequence in a sample having a related reference sequence are provided herein. The kits include a sequencing primer that is complementary to a portion of one strand of the target sequence and the reference sequence and a blocking nucleic acid (BNA) that is fully complementary with at least a portion of one strand of the reference sequence and is not fully complementary with either strand of the target sequence. The sequencing primer and the blocking nucleic acid are complementary to the same strand of the reference sequence and the blocking nucleic acid is blocked at the 3′ end such that it cannot be extended by a polymerase. The kits may also include labeled chain terminating nucleotide triphosphates.

In another aspect, kits for amplifying the target sequence and sequencing the target sequence are also provided. In addition, to the elements in the kits described above, these kits also include a 5′-phosphorylated amplification primer that does not bind the same strand of the target sequence as the sequencing primer. The kits may also include lambda exonuclease to degrade the amplification product comprising the 5′-phosphate.

In yet another aspect, methods for preparing a target sequence in a sample for sequencing are provided. The methods include adding the sample having a reference sequence and also suspected of having one or more target sequences to a DNA sequencing reaction mixture to form a reaction mixture. The DNA sequencing reaction mixture includes a sequencing primer and an excess amount of a blocking nucleic acid. The blocking nucleic acid is fully complementary with at least a portion of one strand of the reference sequence and is not fully complementary with either strand of the target sequence. The blocking nucleic acid is blocked at the 3′ end such that it cannot be extended by a polymerase and both the blocking nucleic acid and the sequencing primer are complementary to the same strand of the reference sequence. The reaction mixture suspected of having the target sequence is subjected to a first denaturing temperature that is above the melting temperature (Tₘ) of the reference sequence and the target sequence to form denatured reference strands and denatured target strands. Then the temperature of the reaction mixture is reduced to permit formation of duplexes of the blocking nucleic acid and the complementary reference strand and heteroduplexes of the blocking sequence and target strands. The reaction mixture is then subjected to a critical temperature (T_{c}) sufficient to preferentially denature said heteroduplexes of the blocking nucleic acid and the complementary target strands, as compared to denaturing duplexes of the blocking nucleic acid and the complementary reference strand. The temperature of the reaction mixture is then reduced to permit the sequencing primer to anneal to free target strands and free reference strands in the reaction mixture. Finally, the sequencing primer is extended to generate extension products which are capable of being analyzed to allow determination of the nucleic acid sequence of the target sequence.

In still another aspect, the target sequence may be amplified using PCR prior to or simultaneously with the sequencing method described above. In one embodiment, one strand of the amplified target sequence may be selectively degraded. Suitably, the degraded strand is the strand complementary to the sequencing primer. In one embodiment, a 5′-phosphorylated amplification primer is added with the sequencing primer to a PCR reaction and the target sequence is amplified. The strand of the amplified target sequence comprising the 5′-phosphate can be degraded by incubation with lambda exonuclease.

Other embodiments and advantages of the invention may be apparent to those skilled in the art upon reviewing the drawings and the following detailed description.

### BRIEF DESCRIPTION OF THE DRAWINGS

FIG. 1 is a depiction of the methods described herein.

FIG. 2 is a set of sequencing electropherograms of K-RAS G12V and wild-type DNA using a reverse M13 primer. The sample contains 85% wild-type and 15% G12V mutation DNA.

FIG. 3 is a set of sequencing electropherograms of K-RAS G12V and wild-type DNA using a forward M 13 primer. The sample contains 85% wild-type and 15% G12V mutation DNA.

FIG. 4 is a set of sequencing electropherograms of K-RAS G12R and wild-type DNA after initial Ice-COLD-PCR of K-RAS G12R followed by BLOCker™ sequencing with the reverse blocking nucleic acid (BNA) and reverse M13 primer. The initial sample for the PCR contains 99% wild-type and 1% G12R mutation DNA. The top panel shows the results of a reaction containing 0 nM BNA in the sequencing reaction, the second panel shows the results of a reaction containing 50 nM BNA, the third panel shows the results of a reaction containing 75 nM BNA and the bottom panel shows the results of a reaction containing 100 nM BNA.

FIG. 5 is a set of sequencing electropherograms of K-RAS G12R and wild-type DNA after initial Ice COLD-PCR of K-RAS G12R followed by BLOCker sequencing with the forward BNA and forward M13 primer. The initial sample for the PCR contains 99% wild-type and 1% G12R mutation DNA. The top panel shows the results of a reaction containing 0 nM BNA in the sequencing reaction, the second panel shows the results of a reaction containing 50 nM BNA, the third panel shows the results of a reaction containing 75 nM BNA and the bottom panel shows the results of a reaction containing 100 nM BNA.

FIG. 6 is a set of sequencing electropherograms of a mitochondrial mutation using reverse primer and reverse BNA as described in Example 4.

FIG. 7 is a set of sequencing electropherograms of HPV18 and HPV45 mixtures using the HPV18F BNA (BNA titration from 0-75 nM, Tc of 75.3° C.).

FIG. 8 is a set of sequencing electropherograms of HPV18 and HPV45 mixtures using the HPV18F BNA (BNA concentration of 75 nM, denaturing temperature (Tc) from 74.2-80.0° C.).

FIG. 9 is a set of sequencing electropherograms of HPV18 and HPV45 mixtures using the HPV45F BNA (BNA titration from 0-75 nM, denaturing temperature (Tc) of 76.3° C.).

FIG. 10 is a set of sequencing electropherograms of HPV18 and HPV45 mixtures using the HPV45F BNA (BNA concentration of 50 nM, denaturing temperature (Tc) from 74.2-80.0° C.).

FIG. 11 is a set of sequencing electropherograms of HPV97 and HPV56 mixtures using the HPV56F BNA (BNA titration from 0, 50, 75, and 100 nM, denaturing temperature (Tc) of 73.3° C.). The dark highlighted portion allowed the alignment of the mixture without the BNA to that of the expected sequence result. The lighter highlighted portions are those where the sequence differs between HPV56 and HPV97.

FIG. 12 is a set of sequencing electropherograms of HPV56 and HPV97 mixtures using the HPV97F BNA (BNA titration from 0, 50, 75, and 100 nM, denaturing temperature (Tc) of 73.3° C.). The dark highlighted portion of the sequence allowed the alignment of the mixture without the BNA to that of the expected sequence result. The lighter highlighted portions of the sequence are those where the sequences differ between HPV56 and HPV97.

FIG. 13 is a set of sequencing electropherograms of HPV56 and HPV97 mixtures using either the HPV97F or HPV56F BNA (BNA concentration of 75 nM, denaturing temperature (Tc) of 73.3° C.) as compared to sequencing without a BNA. The differences in sequence between the two strains are highlighted.

FIG. 14 is a diagram showing the Ice COLD-PCR and BLOCker sequencing strategy including the primers and BNA used for amplifying and sequencing a small amount of the K-RAS exon 2 mutant in the background of a large amount of wild-type K-RAS. The bolded sequence is the K-RAS exon 2 coding region. The two italicized regions indicate the forward and reverse primer locations used in the first round of the PCR. The underlined sequences indicate the locations of the forward and reverse primers used in the ICE COLD PCR amplification reaction. The region in parenthesis indicates the sequence of the BNA with the underlining (C) indicating the positions of incorporation of an LNA. The sequence in light gray indicates the location of the sequencing primer.

FIG. 15 is a set of sequencing electropherograms of BRAF exon 15 showing decreasing amounts of the V600E mutant in the background of wild-type DNA as detected after ICE-COLD PCR, BLOCker Sequencing or standard Sanger sequencing. The arrows indicate the location of the V600E mutation and the limit of detection of the mutant is circled.

FIG. 16 is a set of sequencing electropherograms of BRAF exon 11 showing decreasing amounts of the G469A mutant in the background of wild-type DNA as detected after ICE-COLD PCR and BLOCker Sequencing. The arrows indicate the location of the G469E mutation and the limit of detection of the mutant is circled.

### DETAILED DESCRIPTION

Kits and methods for sequencing a target DNA sequence in a sample having a related reference sequence are provided herein. The kits and methods allow for sequencing of a target sequence in a background of related reference sequences by the addition of a blocking nucleic acid during the sequencing reaction. The kits and methods described herein may also be combined with PCR amplification.

The kits and methods described herein may be used in a variety of situations in which one wants to identify a target nucleic acid from within a mixed population of sequences with some sequence homology. In particular, the kits and methods may be useful for mutation analysis, in particular somatic mutational analysis, and can be used to identify cells or subjects having mutations related to, for example, development of cancer, prognosis of cancer or small molecule and biologic drug efficacy, mosaicism or mitochondrial myopathies. For other potential applications of this method for somatic mutation analysis, see, for example, Erickson R P. (2010) Somatic gene mutation and human disease other than cancer: an update. Mutat Res. 705(2):96-106.

In the Examples, assays for detection of mutations in K-RAS and BRAF known to be associated with cancerous transformation of cells and an assay for detection of mutations in mitochondrial DNA associated with development of MELAS (Mitochondrial Encephaloinyopathy, Lactic Acidosis, and Stroke-like episodes) are demonstrated. The methods and kits may also be used to identify other types of low level mitochondrial heteroplasmy. In addition, the methods and kits are useful for determining strain or species designation in a potentially mixed population, such as during an infection. In the Examples, human papilloma virus (HPV) strains 18 and 45 or strains 57 and 96 were differentiated in a mixed population. The methods could also be used to identify antibiotic resistant mutants developing during drug treatment of an infection, such as in a viral e.g., HIV, or bacterial infection. Those skilled in the art will appreciate other uses of the kits and methods described here.

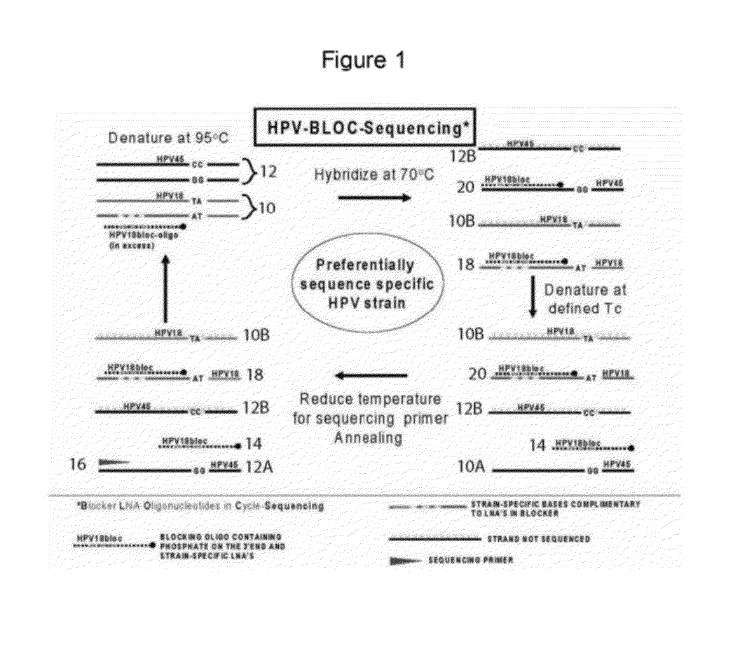

FIG. 1 illustrates preparing a target sequence for sequencing in accordance with the methods and kits of the present invention. To begin (FIG. 1, step 1, upper left corner), the nucleic acid sample contains a double-stranded reference sequence 10 (e.g., a wild-type sequence) and a double-stranded target sequence 12 (e.g., a mutant sequence). The sequencing reaction mixture contains the sample, the sequencing primer 16, other sequencing ingredients such as nucleotide triphosphates (NTPs) some of which may be labeled and strand terminating NTPs or dideoxyNTPs, a DNA polymerase, and a blocking nucleic acid 14 at an excess concentration level, such as 25 nM. Suitably, the blocking nucleic acid is present at a molar excess concentration level as compared to the target and reference sequences.

In FIG. 1, the depicted blocking nucleic acid 14 is a single-stranded nucleic acid sequence complementary with one of the strands 10A of the reference sequence 10. The blocking nucleic acid 14 and the sequencing primer 16 are complementary to the same strand of the reference sequence 10 and the blocking nucleic acid 14 is blocked at the 3′ end such that it cannot be extended by a polymerase.

The reaction mixture in step 1 of FIG. 1 is subjected to a first denaturing temperature, e.g. 95° C. for 15 seconds, which results in denatured strands of the reference sequence 10A, 10B and the target sequence 12A, 12B (to provide reference strands and target strands). The reaction mixture is then cooled to promote hybridization, e.g., 70° C. for 120 seconds. The temperature reduction occurs in the presence of an excess amount of blocking nucleic acid 14, to permit the blocking nucleic acid 14 to preferentially hybridize with the complementary strand 10A of the reference sequence and also the complementary strand 12A of the target sequence. Step 2 in FIG. 1 illustrates the state of the reaction mixture after hybridization at 70° C. In addition to homoduplexes 18 of the blocking nucleic acid 14 and the complementary reference strand 10A and heteroduplexes 20 of the blocking nucleic acid 14 and the complementary target strand 12A, the reaction mixture also contains the denatured negative strands 10B and 12B of the reference and target sequences, respectively. There may also be some complementary strand and target strand homoduplexes as well as complementary strand: target strand heteroduplexes; the excess of blocking nucleic acid in the reaction is designed to minimize the quantities of these complexes.

In step 3 of FIG. 1, the reaction mixture is then subjected to the critical temperature “T_{c}”, e.g., 84.5° C., which is chosen to permit preferential denaturation of the heteroduplexes 20 of the target strand 12A and blocking nucleic acid 14. Suitably, the temperature in step 3 is higher than the temperature used in step 2, such that the temperature is increased to the critical temperature. The critical temperature (T_{c}) is selected so that duplexes 18 of the blocking nucleic acid 14 and the complementary reference strands 10A remain substantially nondenatured when the reaction mixture is incubated at T_{c}. The melting temperature for the duplex 20 of the blocking nucleic acid 14 and the target strand 10B will always be less than the melting temperature of the duplex 18 of the blocking nucleic acid 14 and the complementary reference strand 10A because the blocking nucleic acid 14 is fully complementary with at least a portion of the reference strand 10A, and there will be at least one mismatch with the target strand 12A.

Referring to step 4 of FIG. 1, after preferential denaturation, the temperature of the reaction mixture is reduced, e.g., 50° C., to permit the sequencing primer 16 to anneal to the free target strand 12A in the reaction mixture. Step 4 of FIG. 1 illustrates that the sequencing primer 16 does not bind to the free reference strand 10B or the free target strand 12B, but only to the free target strand 12A. The sequencing primer 16 cannot effectively anneal to the remaining free reference strand 10A or cannot be extended to allow for sequencing of the remaining free reference strand 10A because the reference strand 10A is hybridized with the blocking nucleic acid 14, and at least the section of the reference strand 10A hybridized to the blocking nucleic acid 14 is unavailable for sequencing. The sequencing primer is suitably added to the reaction mixture such that it is present in excess of the blocking nucleic acid, suitably the sequencing primer is present in molar excess to the BNA, so that target strand:sequence primer duplexes form preferentially to target strand:bloc-king nucleic acid sequence duplexes. The temperature of the reaction mixture may then be raised, e.g. 60° C., to extend the annealed sequencing primer 16. Alternatively, a cycle sequencing reaction can be completed by repeating steps 1-4 of FIG. 1 to enrich the extension product. The method illustrated in FIG. 1 can and should be optimized for individual protocols.

Finally, the nucleic acid sequence of the target sequence may be determined using DNA sequencing methods known to those of skill in the art. For example, labeled chain terminating nucleotides may be included in the DNA sequencing reaction mixture to prepare an extended product for Sanger or di-deoxy sequencing. Those of skill in the art will appreciate that other sequencing methods may be used such as Pyrosequencing®, various next generation platforms like 454™ Sequencing, SOLiD™ System, Illumina HiSeq® Systems, or third generation sequencing platforms. A proposed pyrosequencing method would involve the following steps: (1) PCR of target sequence, (2) alkaline denaturation, (3) purify single-strand template, (4) anneal blocking primer at 70° C., (5) raise temperature to Tc, (6) probable washing step to remove any unbound blocking primer, (7) reduce temperature to anneal sequencing primer, (8) cool to room temperature and proceed with a standard Pyrosequencing reaction.

As described above, the kits and methods include a sequencing primer that is complementary to a portion of one strand of the target sequence and the reference sequence. The sequencing primer is a nucleic acid that is fully complementary to a portion of a strand of target sequences and may also be fully complementary to a portion of a strand of the reference sequences. The sequencing primer is capable of annealing to the reference and target strands such that a polymerase can attach and extend the sequencing primer. The sequencing primer is generally DNA, but may be RNA or contain modified nucleotides. Sequencing primers may be designed to have minimal secondary structure and to inhibit reannealing of the reference and target strands. The sequencing primers suitably have an annealing temperature below the critical temperature (T_{c}). Those of skill in the art familiar with sequencing methods are capable of designing sequencing primers for use in the kits and methods. Computer programs are available to those skilled in the art for use in designing suitable sequencing primers and blocking nucleic acids, e.g., Oligo and Primer3.

The target sequence is the sequence that one wants to determine within a mixed or potentially mixed sample including reference sequences. Target sequence refers to a nucleic acid that may be less prevalent in a nucleic acid sample than a corresponding reference sequence. The target sequence may make-up 0.01 to over 99% of the total amount of reference sequence plus target sequence in a sample. The lower limit of detection is based on the sample size, such that the sample must contain at least one amplifiable target sequence in order to be able to sequence the target sequence. As shown in the Examples, the target sequence could be efficiently sequenced using the methods when present at 50%, 15%, 1% or even 0.5% of the total of reference sequence plus target sequence. It is predicted that the methods described herein could be combined with other methods of selective amplification of a target sequence to increase the limit of detection of the target sequence in a background of reference sequences. As shown in the examples, the methods described herein may be used on a sample previously subjected to ICE COLD-PCR as described in International Patent Publication No. WO2011/112534, which is incorporated herein by reference in its entirety. The limit of detection shown in the Examples when ICE COLD PCR was combined with the BLOCker sequencing method described herein is lower than that of either method used on its own. For example, the limit of detection may be lower than 0.01% target in a background of reference sequence. With further optimization we expect the limit of detection could be lowered to the point at which a single copy of the target sequence can be detected in the background of the reference sequence.

The target sequence may include, but is not limited to a somatic mutation, a mitochondrial mutation, a strain or species. For example, a sample (e.g., blood sample) may contain numerous normal cells and few cancerous cells and/or free-circulating tumor DNA. The normal cells contain non-mutant or wild-type alleles, while the small number of cancerous cells and low levels of free-circulating tumor DNA contain somatic mutations. In this case the mutant is the target sequence while the wild-type sequence is the reference sequence. The target sequence must differ by at least one nucleotide from the reference sequence, but must be at least 50% homologous to the corresponding reference sequence. The sequencing primer should be able to bind to both the target sequences and the reference sequences. As used herein, a “target strand” refers to a single nucleic acid strand of a target sequence.

Reference sequence refers to a nucleic acid that is present in a nucleic acid sample and inhibits effective sequencing of a target sequence by traditional sequencing methods without use of a blocking nucleic acid. The reference sequence may make-up 0.01 to 99% or more of the total reference sequence plus target sequence in a sample prior to the use of the method described herein. The lower limit of detection is based on the sample size, such that the sample must contain at least one amplifiable reference sequence in order to be able to sequence the reference sequence. As noted above, the limit of detection may be optimized by combining the methods described herein with other methods such as ICE COLD PCR. As used herein, a “reference strand” refers to a single nucleic acid strand of a reference sequence.

The reference sequence may also be referred to as the wild-type. The term “wild-type” refers to the most common polynucleotide sequence or allele for a certain gene in a population. Generally, the wild-type allele will be obtained from normal cells.

The target sequence may also be referred to as the mutant sequence. The term “mutant” refers to a nucleotide change (i.e., a single or multiple nucleotide substitution, inversion, deletion, or insertion) in a nucleic acid sequence. A nucleic acid which bears a mutation has a nucleic acid sequence (mutant allele) that is different in sequence from that of the corresponding wild-type polynucleotide sequence. The invention is broadly concerned with somatic mutations and polymorphisms. The methods described herein are useful in selectively enriching a target strand which contains 1 or more nucleotide sequence changes as compared to the reference strand. A target sequence will typically be obtained from diseased tissues or cells and may be associated with a disease state or predictive of a disease state or predictive of the efficacy of a given treatment.

The target and reference sequences can be obtained from a variety of sources including, genomic DNA, cDNA, mitochondrial DNA, viral DNA or RNA, mammalian DNA, fetal DNA, parasitic DNA or bacterial DNA. While the reference sequence is generally the wild-type and the target sequence is the mutant, the reverse may also be true. The mutant may include any one or more nucleotide deletions, insertions or alterations. The target sequence may be a sequence indicative of cancer in a cell, metastases of cancer via detection of cells comprising the mutation in a different tissue or in the blood, prognosis of cancer or another disease, drug or chemotherapeutic sensitivity or resistance of a cancer or a microorganism to a therapeutic, or presence of a disease related to a somatic mutation such as mitochondrial heteroplasmy.

The blocking nucleic acid is an engineered single-stranded nucleic acid sequence, such as an oligonucleotide and preferably has a length smaller than the target sequence. The blocking nucleic acid is also suitably smaller than the reference sequence. The blocking nucleic acid must be of a composition that allows differentiation between the melting temperature of duplexes of the blocking nucleic acid and the target strand from that of duplexes of the blocking nucleic acid and the reference strand. The 3′-OH end of the blocking nucleic acid is blocked to DNA-polymerase extension, the 5′-end may also be modified to prevent 5′ to 3′ exonucleolysis by DNA polymerases. The blocking nucleic acid can also take other forms which remain annealed to the reference sequence when the reaction mixture is subject to the critical temperature “T_{c}”, such as a chimera between single stranded DNA, RNA, peptide nucleic acid (PNA), locked nucleic acid (LNA), or another modified nucleotide. PNAs, LNAs or other modified nucleotides in the blocking nucleic acid may be selected to match positions where the reference sequence and the target sequence are suspected to be different. Such a design maximizes the difference between the temperature needed to denature heteroduplexes of the blocking nucleic acid and the partially complementary target strands and the temperature needed to denature duplexes of the blocking nucleic acid and the fully complementary reference strand. Alternatively or in addition, the position of modified nucleotides may be selected to design the blocking nucleic acid to have a more constant melting temperature across the blocking nucleic acid.

The blocking nucleic acid can take many forms, yet the preferred form is single stranded, non-extendable DNA. Suitably the 3′ end of the sequencing primer binds to a position near the 5′ end of the blocking nucleic acid or complementary to at least one of the same bases of the reference sequence as the 5′ end of the blocking nucleic acid. In an alternative embodiment, the sequencing primer overlaps the blocking nucleic acid by 3-5 bases. In this embodiment the DNA polymerase used for sequencing may be a strand-displacing or a non-strand displacing DNA polymerase. In another alternative the sequencing primer and the blocking nucleic acid do not overlap. If the sequencing primer and the blocking nucleic acid do not overlap it is preferable to use a non-strand displacing DNA polymerase for the sequencing reaction. More specifically, the preferred blocking nucleic acid has the following characteristics:
(a) comprises single-stranded nucleic acid;
(b) is fully complementary with at least a portion of the reference sequence;
(c) is complementary to the same strand of the reference sequence as the sequencing primer; and
(d) contains a 3′-end that is blocked to DNA-polymerase extension.

The blocking nucleic acid can be synthesized in one of several methods. First, the blocking nucleic acid can be made by direct synthesis using standard oligonucleotide synthesis methods that allow modification of the 3′-end of the sequence. Alternatively, the blocking nucleic acid can be made by polymerase synthesis during a PCR reaction that generates single stranded DNA as the end product. In this case, the generated single-stranded DNA corresponds to the exact sequence necessary for the blocking nucleic acid. Methods to synthesize single stranded DNA via polymerase synthesis are well known to those skilled in the art. Alternatively, a single-stranded blocking nucleic acid can be synthesized by binding double-stranded PCR product on solid support. This is accomplished by performing a standard PCR reaction, using a primer pair one of which is biotinylated. Following PCR, the PCR product is incubated with a Streptavidin-coated solid support (e.g. magnetic beads) and allowed to bind to the beads. Subsequently, the temperature is raised to 95° C. for 2-3 minutes to denature DNA and release to the solution the non-biotinylated DNA strand from the immobilized PCR product. The magnetic beads with the complementary DNA strand are then removed and the single-stranded product remaining in the solution serves as the blocking nucleic acid.

Before the single-stranded blocking nucleic acid is used, the 3′-end is blocked to prevent polymerase extension. The 3′-end may contain a phosphate group, an amino-group, a dideoxynucleotide or any other moiety that blocks 5′ to 3′ polymerase extension. This can be accomplished in several ways well known to those skilled in the art. For example, a reaction with Terminal Deoxynucleotide Transferase (TdT) can be employed, in the presence of dideoxynucleotides (ddNTP) in the solution, to add a single ddNTP to the end of the single-stranded blocking nucleic acid. ddNTPs serve to block polymerase extension. Alternatively, an oligonucleotide template complementary to the 3′-end of the blocking nucleic acid can be used to provide a transient double-stranded structure. Then, polymerase can be used to insert a single ddNTP at the 3′-end of the blocking nucleic acid opposite the hybridized oligonucleotide.

The blocking nucleic acid should be present in excess of the amount of reference strands plus target strands (i.e., a molar excess). The required amount of blocking nucleic acid may be determined empirically by those of skill in the art. Generally the amount of blocking nucleic acid is in excess of 5 nM. The Examples provide data using 25 nM, 50 nM, 75 nM and 100 nM blocking nucleic acid in protocols. Generally the sequencing primer should be added such that it is present in the reaction mixture in molar excess concentration as compared to the blocking nucleic acid.

The melting temperature or “Tₘ” refers to the temperature at which a polynucleotide dissociates from its complementary sequence. Generally, the Tₘ may be defined as the temperature at which one-half of the Watson-Crick base pairs in a double-stranded nucleic acid molecule are broken or dissociated (i.e., are “melted”) while the other half of the Watson-Crick base pairs remain intact in a double-stranded conformation. In other words, the Tₘ is defined as the temperature at which 50% of the nucleotides of two complementary sequences are annealed (double-strands) and 50% of the nucleotides are denatured (single-strands). Tₘ, therefore defines a midpoint in the transition from double-stranded to single-stranded nucleic acid molecules (or, conversely, in the transition from single-stranded to double-stranded nucleic acid molecules).

The Tₘ can be estimated by a number of methods, for example by a nearest-neighbor calculation as per Wetmur 1991 (Wetmur, J. G. 1991. DNA probes: applications of the principles of nucleic acid hybridization. Crit Rev Biochem Mol Biol 26: 227-259,) and by commercial programs including Oligo™ Primer Design and programs available on the internet. Alternatively, the Tₘ can be determined through actual experimentation. For example, double-stranded DNA binding or intercalating dyes, such as Ethidium bromide or SYBR®-green (Molecular Probes) can be used in a melting curve assay to determine the actual Tₘ of the nucleic acid. Additional methods for determining the Tₘ of a nucleic acid are well known in the art.

The term “critical temperature” or “T_{c}” refers to a temperature selected to preferentially denature duplexes of target strands and the blocking nucleic acid. The critical temperature (T_{c}) is selected so that duplexes consisting of the blocking nucleic acid and complementary reference strands remain substantially nondenatured when the reaction mixture is incubated at T_{c}, yet duplexes consisting of the blocking nucleic acid and the target strands substantially denature. The term “substantially” means at least 60%, and preferably at least 90% or more preferably at least 98% in a given denatured or nondenatured form.

### Samples

Samples include any substance containing or presumed to contain a nucleic acid of interest (target and reference sequences) or which is itself a nucleic acid containing or presumed to contain a target nucleic acid of interest. The term sample thus includes a sample of nucleic acid (genomic DNA, cDNA, RNA), cell, organism, tissue, fluid, or substance including, but not limited to, for example, plasma, serum, spinal fluid, lymph fluid, synovial fluid, urine, tears, stool, external secretions of the skin, respiratory, intestinal and genitourinary tracts, saliva, blood cells, biopsy, tumors, organs, tissue, samples of in vitro cell culture constituents, natural isolates (such as drinking water, seawater, solid materials), microbial specimens, and objects or specimens that have been “marked” with nucleic acid tracer molecules.

Nucleic acid sequences of the invention can be amplified, e.g., by polymerase chain reaction, prior to use in the methods described herein. The amplification products may be directly sequenced by selectively degrading one strand of the amplified target sequence. One method of selecting a single strand of a double-stranded DNA product is described above in regard to preparation of a single stranded blocking nucleic acid, i.e. one strand may be biotinylated and bound to a column or solid support coated with streptavidin. The non-biotinylated strands can then be purified by denaturing the strands and removing the biotinylated strand bound to the avidin coated solid support in order to allow for sequencing of the non-biotinylated strand. Alternatively, as described in the examples the PCR reaction can be carried out using a 5′-phosphorylated amplification primer in addition to the sequencing primer such that one strand of the product comprises a 5′ phosphate. This strand can then be degraded by incubation with a 5′-phosphate dependent exonuclease, such as lambda exonuclease which was used in the Examples.

The nucleic acid sequences may be from RNA, mRNA, cDNA and/or genomic DNA. These nucleic acids can be isolated from tissues or cells according methods known to those of skill in the art. Complementary DNA or cDNA may also be generated according to methods known to those of skill in the art. Alternatively nucleic acids sequences of the invention can be isolated from blood by methods well known in the art.

As shown in the Examples, methods and kits capable of detecting and sequencing K-RAS exon 2, codon 12 and/or 13 mutations are provided. Detection of these mutations is important to determine the prognosis for subjects with cancer as well as to determine the presence or emergence of drug resistant tumor cells. Epidermal growth factor receptor (EGFR) antagonists, such as cetuximab and panitumumab, are therapeutic agents that can be effective in colorectal cancer (CRC) treatment. It has been shown that 40% of CRC tumors have activating K-RAS exon 2 codon 12 and 13 mutations and that these mutations may be associated with a poor response to EGFR antagonists. Very high sensitivity detection of such diagnostic biomarkers is necessary to determine the presence or emergence of drug resistant tumor cell populations.

In the Examples, a blocking nucleic acid was used to allow sequencing and identification of a known mitochondrial mutation at position 3243 (A→G). This mutation is one of the nine confirmed MELAS (Mitochondrial Encephalomyopathy, Lactic Acidosis, and Stroke-like episodes) mutations in the mitochondrial genome. Thus, the methods and kits of the invention can be used to identify subjects having a low level of a mutation associated with a disease.

Also in the Examples, the methods are employed to differentiate between strains of HPV. The Examples demonstrate that samples comprising mixtures of HPV18 and 45 or of HPV56 and 97 can be differentiated. Such strain differentiation may be important for epidemiological studies and may effect treatment decisions.

The Examples also demonstrate that the methods can be used to detect two BRAF mutations (V600E (exon 15) and G469A (exon 11)) with a limit of detection of 0.5%. These BRAF mutations are associated with cancer, in particular melanoma. As described above for K-RAS, detection of these mutations is important to determine the prognosis for subjects with cancer and may prove relevant for determination of chemotherapeutic effectiveness.

The following examples are meant only to be illustrative and are not meant as limitations on the scope of the invention or of the appended claims. All references cited herein are hereby incorporated by reference in their entireties.

### EXAMPLES

### Example 1

### K-RAS BLOCker Sequencing after Standard PCR using The K-RAS Exon 2 Reverse BNA

Mutations in K-RAS exon 2, codon 12 and 13 are found in several cancers and are associated with resistance to certain anti-cancer drugs. Thus assays to identify samples or subjects comprising these K-RAS mutations would be beneficial. Often these imitations are difficult to identify because the populations are mixed.

Blocking nucleic acids (BNA) were designed to specifically bind to the wild-type K-RAS sequence and unless otherwise noted were made by Exiqon. The BNA and sequencing primer used for this experiment were as follows:

| | BNA | T_{c}(° C.) | Sequencing Primer |
|---|---|---|---|
| K-RASe2 | CTGGTGGCGTAGGCAAGAGTGCCTTG | 81.0 | ATGGTCATAGCTGTTTCCT |
| Reverse | ACGATACAGCTAATTCAGA/3Phos/ | | (SEQ ID NO: 2) |
| | (SEQ ID NO: 1) | | |

wherein the underlined nucleotides are LNAs and the other nucleotides are traditional nucleotides. There was no overlap between the BNA and the sequencing primer.

The nucleic acid samples were prepared using standard protocols and the nucleic acid containing the codon 12 mutation (K-RAS G12V;GTT; 5′-CGCCAACAGCT-3′; SEQ ID NO: 3; underlined base is site of mutation) represented 15% of the total nucleic acid and the remaining 85% of the sample was wild-type genomic DNA (GGT; 5′-CGCCACCAGCT-3′; SEQ ID NO: 4; underlined base is site of mutation). The BNA (25 nM) and nucleic acid were added to a standard cycle sequencing reaction mix.

The sequencing reaction mixture was denatured at 95° C. for 15 seconds, then the temperature was reduced to 70° C. for 45 seconds to allow hybridization of the BNA to the reference strands and target strands. The reaction mixture was then subjected to the Tc of 81° C. for 30 seconds to allow the duplexes of the BNA and target strands to denature. The reaction mixture was then subjected to a temperature of 50° C. for 10 seconds to allow the sequencing primer to anneal to the free target strands. Finally extension of the sequencing primer was allowed to proceed at 60° C. for 25 seconds to generate extension products. The above cycle was repeated 40 times to generate enough sequence to be read on an ABI Sequencer.

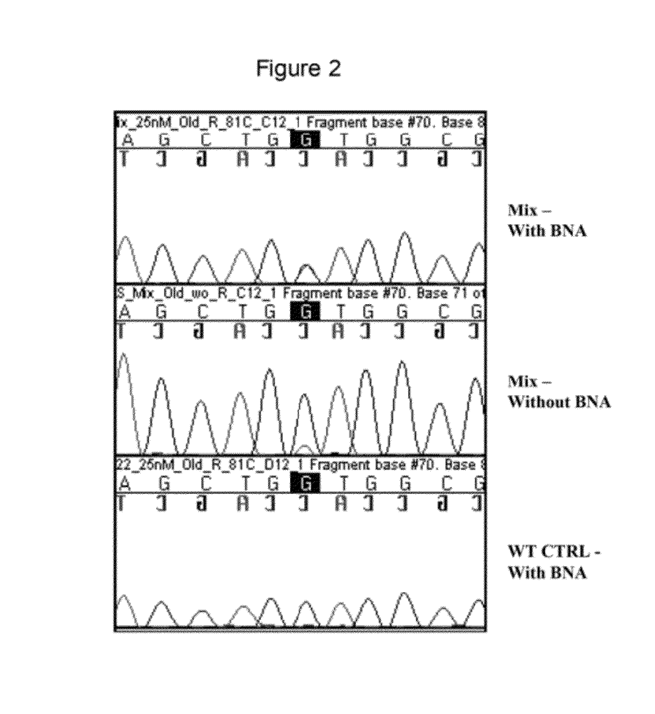

As shown in FIG. 2, the G12V K-RAS mutant was difficult to detect when present in 15% of the total in a sequencing reaction without the BNA (see small peak at highlighted base in middle trace), but detection was increased when the sequencing reaction contained a BNA directed to the wild-type sequence (the two peaks now are present in relatively equal amounts in the top trace). Notably the inclusion of the BNA in a sequencing reaction with only wild-type did not completely block the ability to sequence, but only reduced the size (magnitude) of the peak.

### Example 2

### K-RAS BLOCker Sequencing after Standard PCR using the K-RAS Exon 2 Forward BNA

A blocking nucleic acid (BNA) was designed to bind specifically to the opposite strand of the wild-type K-RAS sequence as well. The BNA and the sequencing primer used for this experiment were as follows:

| | BNA | T_{c}(° C.) | Sequencing Primer |
|---|---|---|---|
| K-RASe2 | GCTGAAAATGACTGAATATAAACTTGTG | 77.0 | TGTAAAACGACGGCCAGT |
| Forward | GTAGTTGGAGCTGGTGGCGTA/3Phos/ | | (SEQ ID NO: 6) |
| | (SEQ ID NO: 5) | | |

wherein the underlined nucleotides are LNAs and the other nucleotides are traditional nucleotides. There was no overlap between the BNA and the sequencing primer.

The nucleic acid samples were prepared using standard protocols and the nucleic acid containing the codon 12 mutation (K-RAS G12V; 5′-AGCTGTTGGCG-3′; SEQ ID NO: 7; underlined base is site of mutation) represented 15% of the total nucleic acid and the remaining 85% of the sample was wild-type genomic DNA (5′-AGCTGGTGGCG-3′; SEQ ID NO: 8; underlined base is site of mutation). The BNA (25 nM) and nucleic acid were added to a standard cycle sequencing reaction mix. The cycle sequencing reaction was completed as described above in Example 1. Thus, cycle sequencing can be used for bi-directional sequencing via design of BNAs specific for each strand of the reference sequence.

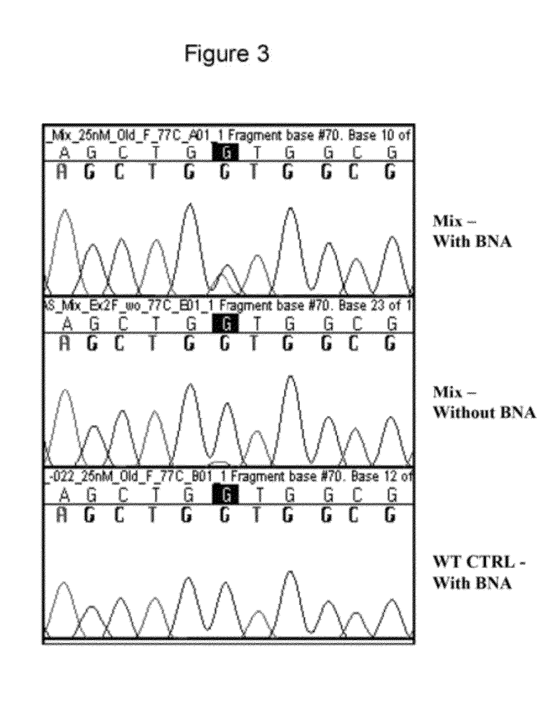

As shown in FIG. 3, the G12V K-RAS mutant was difficult to detect when present in 15% of the total in a sequencing reaction without the BNA (see small peak at highlighted base in middle trace), but detection was increased when the sequencing reaction contained a BNA directed to the wild-type sequence (the two peaks now are visibly present in the top trace). Notably the inclusion of the BNA in a sequencing reaction with only wild-type again did not completely block the ability to sequence, but only reduced the size (magnitude) of the peak.

### Example 3

### K-RAS BLOCker Sequencing Example—After COLD-PCR Detection of the K-RAS G12R Mutation

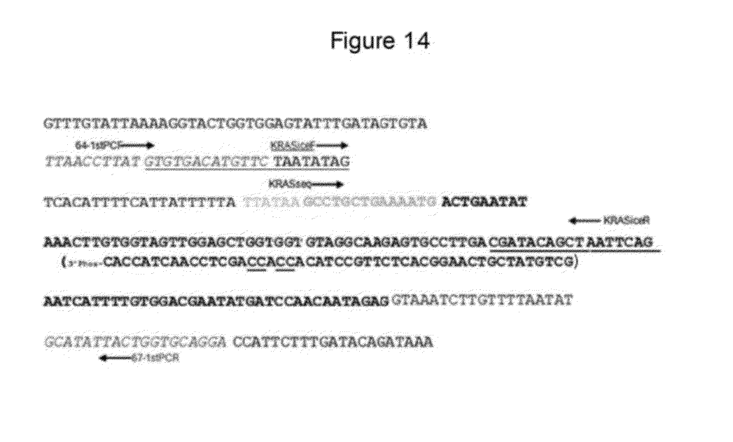

Recently, Ice COLD-PCR (Improved and Complete Enrichment CO-amplification at Lower Denaturation temperature PCR; Milbury et al., Nucleic Acids Res. 2011 Jan. 1; 39(1):e2.) has been shown to improve drastically the detection limit of K-RAS exon 2 mutations. See also International Patent Publication No. WO2011/112534. In Ice COLD-PCR, mutant DNA (Mut) is amplified preferentially in the presence of wild-type (WT) DNA. The use of a reference sequence oligonucleotide (RS-oligo) complementary to one of the WT strands results in linear amplification of the WT sequences but exponential amplification of any Mut sequences present. The RS-oligos may contain Locked Nucleic Acids (LNA™) which increases the difference in denaturation temperature between the RS-oligo:WT DNA duplex as compared to the RS-oligo:Mut DNA duplex. The PCR was carried out as described by Milbury et al. using Phusion® Polymerase in the first round PCR and Optimase in the Ice COLD-PCR. See FIG. 14 (SEQ ID NO: 14) for a diagram depicting the location of the primers and RS-oligo used for Ice COLD-PCR within the K-RAS sequence. The primers and RS-oligo used are as follows:

| | | SEQ |
|---|---|---|
| | | ID |
| USE OF OLIGO | PRIMER | NO: |
| 1^{st} round PCR | 5′-TTAACCTTATGTGTGACATGTTC | 9 |
| forward primer | | |
| 1^{st} round PCR | 5′-TCCTGCACCAGTAATATGC | 10 |
| reverse primer | | |
| ICE COLD | 5′-GTGTGACATGTTCTAATATAG | 11 |
| forward primer | | |
| ICE COLD | 5′-CTGAATTAGCTGTATCG | 12 |
| reverse primer | | |
| RS-oligo for | 5′-GCTGTATCGTCAAGGCACTCTTGC | 13 |
| ICE COLD | CTACACCACCAGCTCCAACTACCAC | |

To further the limit of detection of Ice COLD-PCR, the use of the BNA is expanded to the cycle sequencing reaction. Here, the LNA-containing oligo (BNA) blocks the sequencing of the wild-type DNA and allows the sequencing of DNA containing any mutation (BLOCker-Sequencing). For the blocking to occur, an additional hybridization step as well as a denaturing step (at critical temperature, Tc) is added to the cycle sequencing steps. The Tc is a temperature at which the BNA:WT DNA complex remains intact but the BNA:Mut DNA complex is denatured. The sequencing primer, which overlaps the 5′ end of the BNA in this example, then anneals to the mutant sequence and is subsequently extended.

A blocking nucleic acid (BNA) was designed to specifically bind to the wild-type K-RAS sequence. The BNAs and sequencing primers used for this experiment were as follows:

| | BNA | T_{c}(° C.) | Sequencing Primer |
|---|---|---|---|
| Forward | GAAAATGACTGAATATAAACTTGTG | 77.6 | TTATTATAAGGCCTGCTGAAAATG |
| | GTAGTTGGAGCTGGTGGCGTAGGCA/3Phos/ | | (SEQ ID NO: 16) |
| | (SEQ ID NO: 15) | | |
| Reverse | TTCTGAATTAGCTGTATCGTCAAGG | 82.0 | TATTCGTCCACAAAATGATTCTG |
| | CACTCTTGCCTACGCCACCAGCTCC/3Phos/ | | (SEQ ID NO: 18) |
| | (SEQ ID NO: 17) | | |

wherein the underlined nucleotides are LNAs and the other nucleotides are traditional nucleotides. The italicized bases represent the overlap between the sequencing primer and the BNA.

The nucleic acid samples were prepared using standard protocols and the nucleic acid containing the codon 12 mutation (K-RAS G12R; 5′-GCCACG/CAGCTC-3′ (SEQ ID NO: 19) and 5′-GAGCTC/GGTGGC-3′ (SEQ ID NO: 20)); the underlined bases indicate the site of mutation with the target or mutant sequence listed first and the wild-type sequence after the slash) represented 1% of the total nucleic acid added to the initial PCR experiment and the remaining 99% of the sample was wild-type genomic DNA. The BNA (50 nM, 75 nM or 100 nM) and nucleic acid from the Ice COLD-PCR reaction were added to a standard cycle sequencing reaction mix. The cycle sequencing reaction was completed as described above in Example 1, except that the hybridization time was 120 seconds and the cycle sequencing extension time was 45 seconds. Thus the methods of the current invention can be combined with a PCR enrichment method.

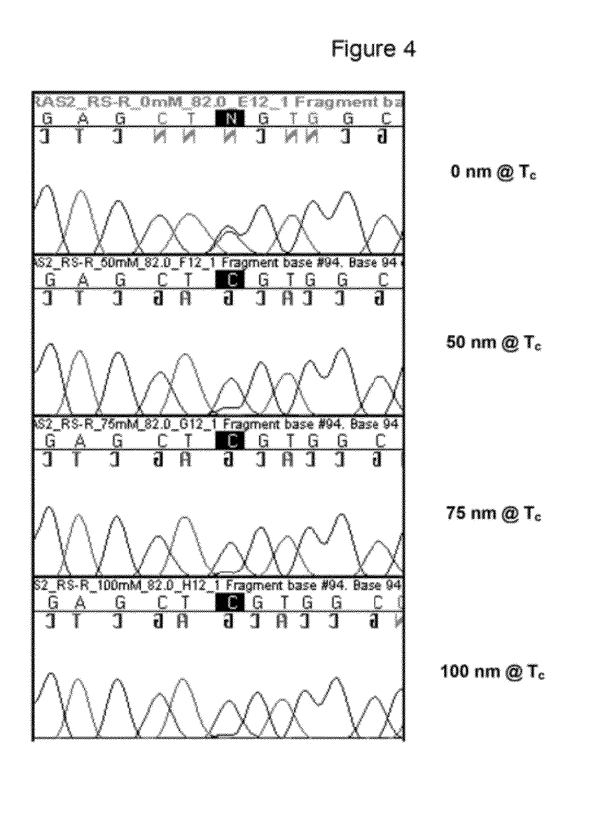

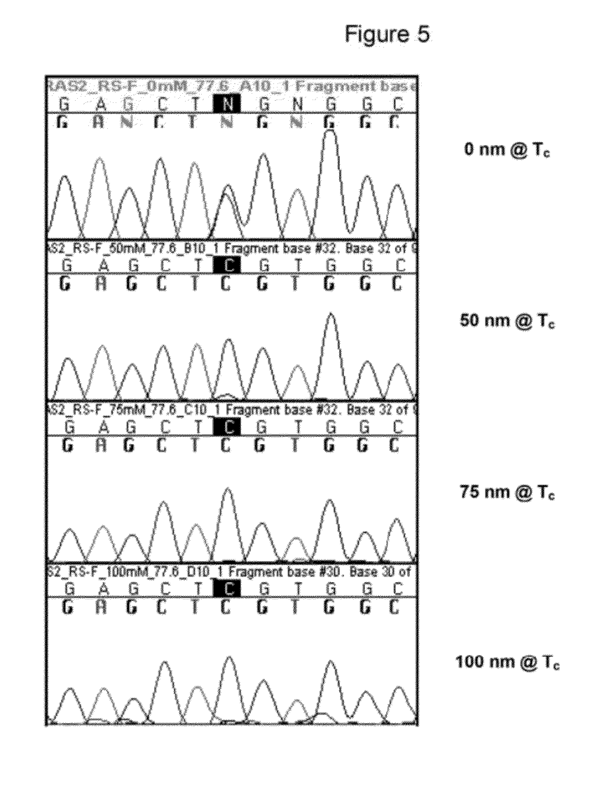

As shown in FIGS. 4 and 5, the K-RAS mutant was difficult to detect in a sequencing reaction without the BNA even after Ice COLD-PCR when present at only 1% of the total sequence (0 nM; see dual peaks at highlighted base in top trace), but detection was increased when the sequencing reaction contained a BNA directed to the wild-type sequence (the larger peak represents the mutant sequence in each of the next three traces).

### Example 4

### Detection of Mitochondrial Somatic Mutations

BLOCker sequencing was performed on a sample with a known mitochondria] mutation at position 3243 (A→G). This mutation is one of the nine confirmed MELAS (Mitochondrial Encephalomyopathy, Lactic Acidosis, and Stroke-like episodes) mutations in the mitochondrial genome. The example below reflects sequencing in the reverse direction using the reverse blocking nucleic acid.

A blocking nucleic acid (BNA) was designed to specifically bind to the wild-type mitochondrial sequence. The BNA and sequencing primers used for this experiment were as follows:

| | BNA | T_{c}(° C.) | Sequencing Primer |
|---|---|---|---|
| 3243 | CCTCTGACTGTAAAGTTTTAAGTTTT | 79.0 | TGTTGTTAAGAAGAGGAATTGAACCTC |
| Forward | ATGCGATTACCGGGCTCTG/3Phos/ | | (SEQ ID NO: 22) |
| | (SEQ ID NO: 21) | | |

wherein the underlined nucleotides are LNAs and the other nucleotides are traditional nucleotides. There was a 4 base overlap between the BNA and the sequencing primer which are shown in italics.

The nucleic acid samples were prepared using standard, protocols and the nucleic acid containing the mutation (5′-GGCAGGGCCCG; SEQ ID NO: 23; mutation underlined) represented 10% of the total nucleic acid and the remaining 90% of the sample was wild-type genomic DNA (5′-GGCAGAGCCCG; SEQ ID NO: 24; wild-type base underlined). The BNA (15 and 25 nM) and nucleic acid were added to a standard cycle sequencing reaction mix. The cycle sequencing reaction was completed as described above in Example 1 with the hybridization time being 120 seconds and the cycle sequencing reaction extension time was 45 seconds, with the total number of cycles increased to 50.

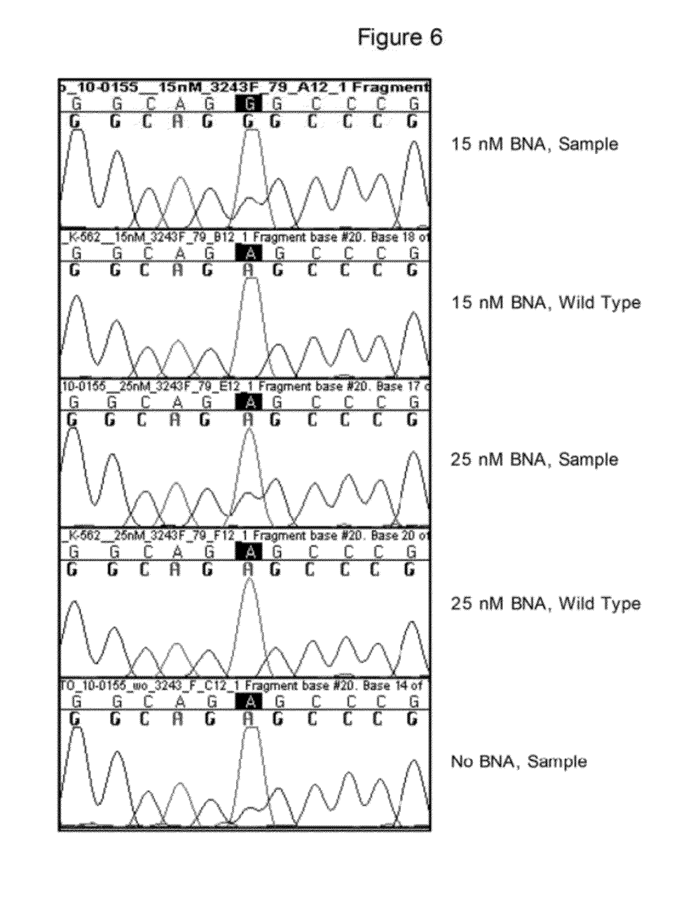

As shown in FIG. 6, the mitochondrial mutant was difficult to detect in a sequencing reaction without the BNA (see small peak at highlighted base in bottom trace), but detection was increased when the sequencing reaction contained a BNA to block the wild-type sequence (see top and third from top trace). The increased presence of the G peak (black) as compared to the sequencing of the sample without LNA shows the improvement of the readability of the mutation. The second, fourth and bottom trace show the wild-type sequence was readily sequenced in the absence or presence of the BNA.

### Example 5

### Sequencing to Differentiate HPV Strains 18 and 45

HPV often presents as a mixed infection of various strains. To identify which strains are present in a sample requires DNA sequencing the strains. Due to the relatively small number of nucleotide changes between the various strains and lack of ability to determine which strains are present in any one sample, it would be beneficial to design a sequencing reaction that could distinguish between strains.

Blocking nucleic acids (BNA) were designed to specifically bind to either the HPV 18 or HPV 45 sequence. The BNAs and sequencing primers used for this experiment were as follows:

| | BNA | T_{c}(° C.) | Sequencing Primer |
|---|---|---|---|
| HPV18 | TTTTTGCAGATGGCTTTGTGGCGGCC | See | CGATCGTAAACGTGTTCCCTATTTTT |
| Forward | TAGTGACAATACCGTATATC/3Phos/ | FIGURE | (SEQ ID NO: 27) |
| | (SEQ ID NO: 25) | | |
| HPV45 | TTTTTGCAGATGGCTTTGTGGCGGCC | | |
| Forward | TAGTGACAGTACGGTATATC/3Phos/ | | |
| | (SEQ ID NO: 26) | | |

wherein the underlined nucleotides are LNAs and the other nucleotides are traditional nucleotides. There was a 3 base overlap between the BNA and the sequencing primer shown in italics.

Stock plasmids (clones of HPV strain templates) were used (10,000 copies/μL) in the experiments described herein. The nucleic acid samples were prepared using standard protocols and amplified by PCR using the Stratagene Brilliant® II Master Mix. Primers used for initial amplification are consensus primers in the L1 region of HPV. A universal tag (UP) was added to both the forward and reverse primer (shaded regions) in order to develop specific sequencing based primers (see Table 1). Table 1 HPV Consensus Primer Sequences (UP1 highlighted in forward primer; UP2 highlighted in reverse primer)

| | |
|---|---|
| HPV | tcgaggtcgacggtatcgatCGTAAACGTTTTCCCTATTTTTTT |
| Cons | (SEQ ID NO: 28) |
| w/UP | |
| F | |
| HPV | 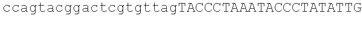 |
| Cons | (SEQ ID NO: 29) |
| w/UP | |
| R | |

After PCR the nucleic acids were mixed such that the HPV 18 nucleic acid represented 50% of the total nucleic acid and the remaining 50% of the sample was HPV45 DNA. The BNA (50 nM for HPV18 and 75 nM for HPV45) and nucleic acid were then added to a standard cycle sequencing reaction mix. The cycle sequencing reaction was completed as described above in Example 1 with the hybridization time being 120 seconds and the cycle sequencing reaction extension time was 45 seconds.

To determine the T_{c} for each BNA, various concentrations of BNA are cycle sequenced using a temperature gradient spanning the calculated Tm of the BNA-with its reference sequence. Each sequencing reaction is evaluated using the sequencing electropherograms for the presence of peaks for both strains and then the preferential disappearance of the reference sequence peak in the sample which is being blocked from sequencing by the BNA. A specific concentration and T_{c} for the BNA is then determined and can be used in the future for preferential cycle sequencing of this mixed sample population.

Various concentrations of the HPV18 BNA were used along with a gradient thermal cycler to determine the critical temperature at which the HPV18 BNA remains duplexed with the HPV18 strain while allowing sequence analysis of HPV45. In the second set of experiments, an HPV45 BNA was used to preferentially sequence HPV18 while blocking sequencing of HPV45.

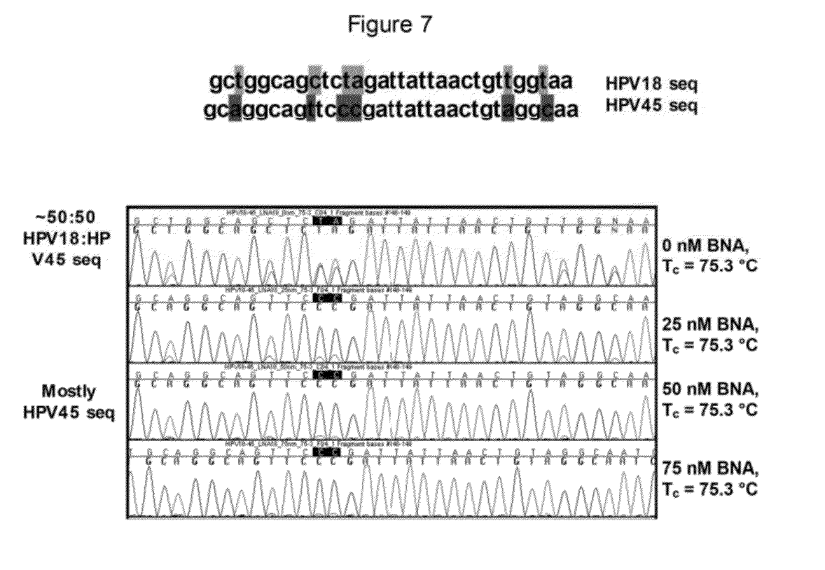

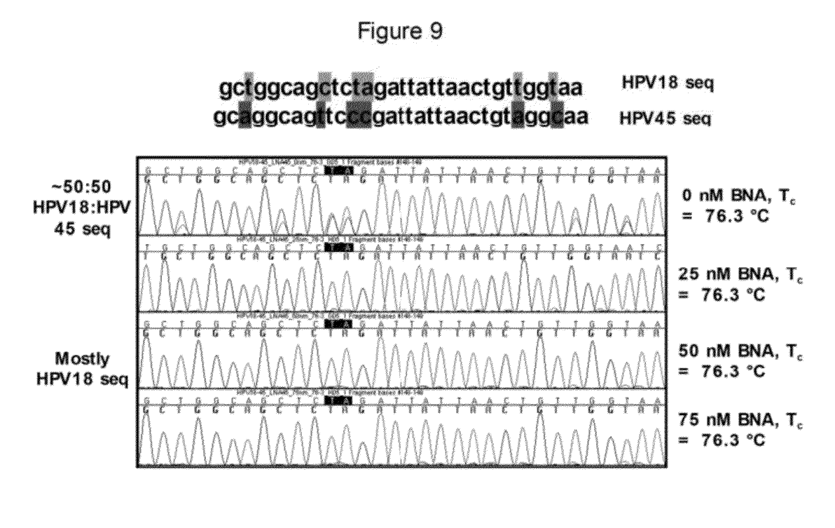

As shown in FIGS. 7 and 9 respectively, the HPV18 (SEQ ID NO: 30 as shown in FIG. 7-10) and HPV 45 (SEQ ID NO: 31 as shown in FIG. 7-10) strains were difficult to sequence without the BNA (see overlapping peaks at highlighted bases in the top trace), but detection of the target sequence was increased when the sequencing reaction contained a BNA to block the reference sequence (the HPV45 sequences become the dominant peaks in the lower traces as more HPV18-specific BNA was added and vice versa in FIGS. 7 and 9, respectively).

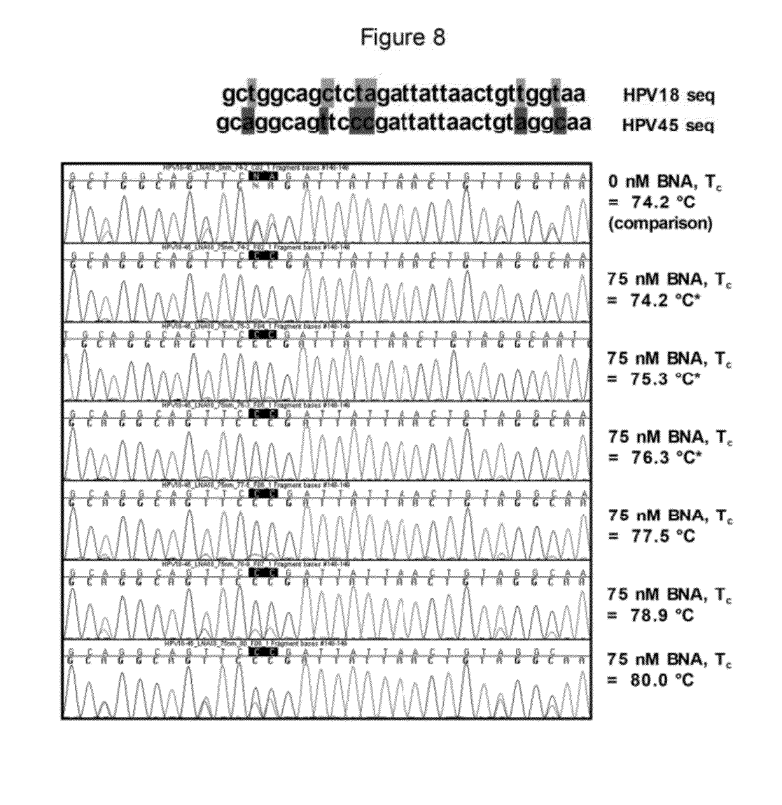

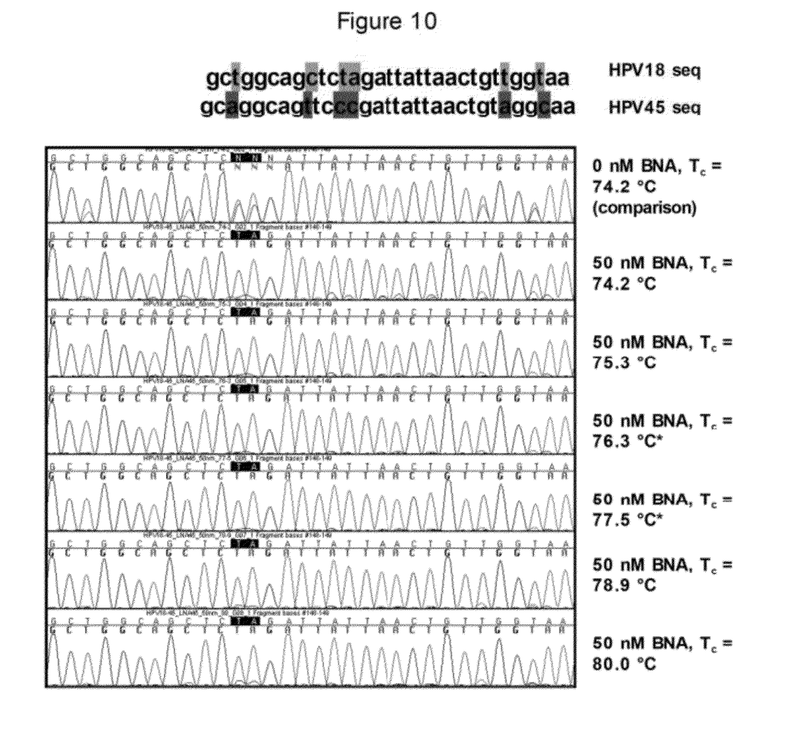

FIGS. 8 and 10 show the effect of altering the temperature at which denaturation of the BNA from the opposing strain should occur. As shown in the top trace without a BNA is unclear. A denaturation temperature that is too low will not block sequencing of the reference sequence and both peaks can be seen. As the temperature is increased in the middle traces the target sequence becomes the dominant peaks. In the bottom trace, the temperature was raised above the T_{c} and allowed sequencing of the reference sequences and mixed peaks again. This example demonstrates that both the amount of the BNA and the temperature selected for denaturation can be selected empirically.

### Example 6

### Sequencing to Differentiate HPV Strains 56 and 97

Blocking nucleic acids (BNA) were designed to bind specifically to either the HPV 56 or HPV 97 sequence. The BNAs and sequencing primers used for this experiment were as follows:

| | BNA | T_{c}(° C.) | Sequencing Primer |
|---|---|---|---|
| HPV56 | TTTTTGCAGATGGCGACGTGGCGGCCTAG | 73.3 | CGATCGTAAACGTGTTCCCTATTTTT |
| Forward | TGAAAATAAGGTGTATCTACC/3Phos/ | | (SEQ ID NO: 34) |
| | (SEQ ID NO: 32) | | |
| HPV97 | TTTTTGCAGATGGCTTACTGGCGGCCTAG | | |
| Forward | TGACAGTACGGTTTATCTGCC/3Phos/ | | |
| | (SEQ ID NO: 33) | | |

wherein the underlined nucleotides are LNAs and the other nucleotides are traditional nucleotides. There was a 3 base overlap between the BNA and the sequencing primer shown in italics.

Stock plasmids (clones of HPV strain templates) were used (10,000 copies/μL) in the experiments described herein. The nucleic acid samples were prepared using standard protocols and amplified by PCR using the Stratagene Brilliant II Master Mix. Primers used for initial amplification are consensus primers in the L1 region of HPV. A universal tag (UP) was added to both the forward and reverse primer in order to develop specific sequencing based primers (see Table 1).

After PCR the nucleic acids were mixed such that the HPV56 nucleic acid represented 50% of the total nucleic acid and the remaining 50% of the sample was HPV97 DNA. The BNA (75 nM for both HPV56 and HPV97) and nucleic acid were then added to a standard cycle sequencing reaction mix. The cycle sequencing reaction was completed as described above in Example 1 with the hybridization time being 120 seconds and the cycle sequencing reaction extension time was 45 seconds.

To determine the T_{c} for each BNA, various concentrations of BNA are cycle sequenced using a temperature gradient spanning the calculated Tm of the BNA-with its reference sequence. Each sequencing reaction is evaluated using the sequencing electropherograms for the presence of peaks for both strains and then the preferential disappearance of the reference sequence peak in the sample which is being blocked from sequencing by the BNA. A specific concentration and T_{c} for the BNA is then determined and can be used in the future for preferential cycle sequencing of this mixed sample population.

Various concentrations of the HPV56 BNA were used along with a gradient thermal cycler to determine the critical temperature at which the HPV56 BNA remains intact with the HPV56 strain while allowing sequence analysis of HPV97. In the second set of experiments, a HPV97 BNA was used to preferentially sequence HPV56 while blocking sequencing of HPV97.

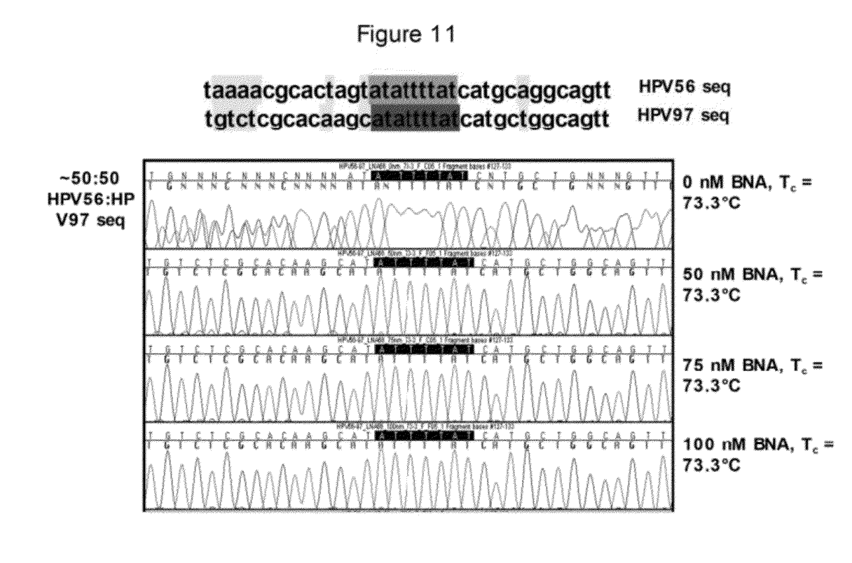

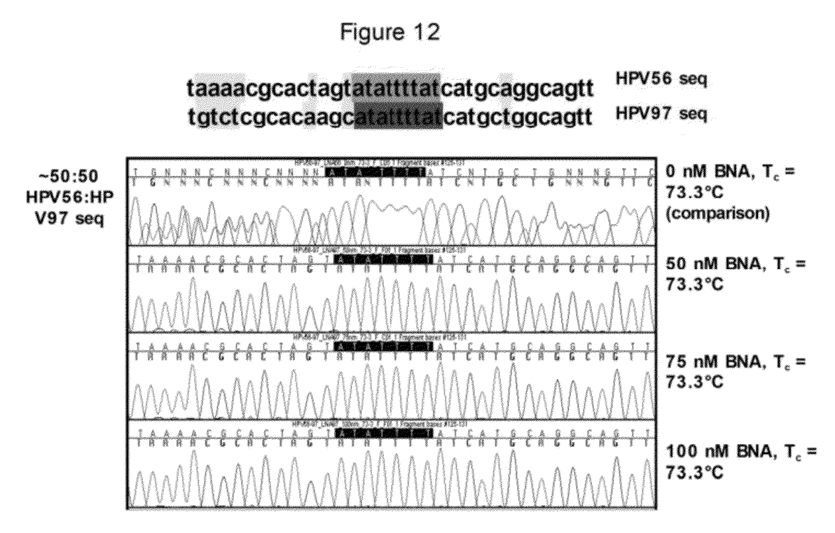

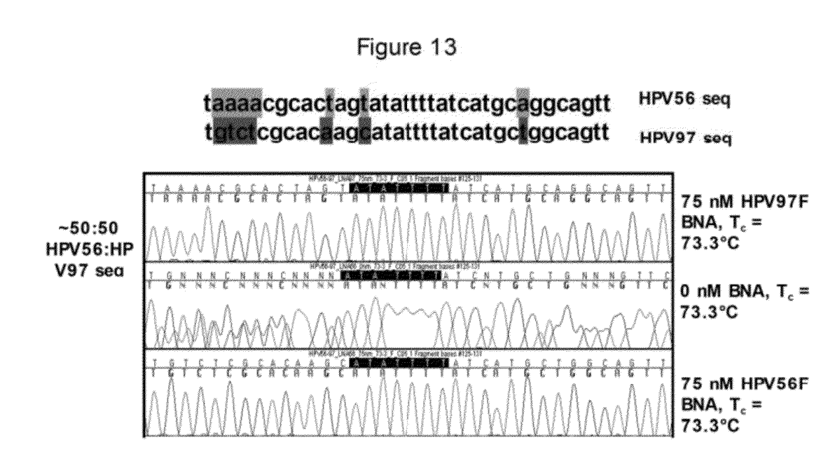

As shown in FIGS. 11 and 12 respectively, the HPV 97 (SEQ ID NO: 35) and HPV 56 (SEQ ID NO: 36) strains were difficult to sequence without the BNA (see overlapping peaks in the top trace), but detection of the target sequence was increased when the sequencing reaction contained a BNA to block the reference sequence (the HPV97 sequences become the dominant peaks in the lower traces as more HPV56-specific BNA was added and vice versa in FIGS. 11 and 12, respectively). FIG. 13 shows the electropherograms of a sequencing reaction with no BNA (middle trace with many areas that are not readable) as compared to the traces obtained using the optimal concentration of BNA and denaturation temperatures (top trace and bottom trace showing resolved sequences for HPV 56 and 97, respectively).

### Example 7

### Amplification followed by Sequencing to Detect a BRAF Mutation

Blocking nucleic acids and primers were designed to specifically amplify and allow for sequencing of two BRAF mutations, V600E and G469A. The sequencing primer was also used as an amplification primer during PCR. The sequencing primer and the BNA were designed to bind to the same strand of the DNA. The amplification primer was designed to bind the opposite or complementary strand and was 5′ phosphorylated.

For detection of BRAF V600E, the primers or oligonucleotides have the following sequences and modifications:

sequencing primer¹
(SEQ ID NO: 37)
5′-ATGCTCAGACACAATTAGCGCGACCCTTAGATCCAGACAACTGTTC
AAAC-3′
5′-phosphorylated amplification primer²
(SEQ ID NO: 38)
/5Phos/TCCTTTACTTACTACACCTCAG-3′
Blocking oligo (BNA)³
(SEQ ID NO: 39)
5′-AACTGTTCAAACTGATGGGACCCACTCCATCGAGATTT+C+A+C+T
GTAGCTAG/3Phos/

For BRAF G469A, the primers or oligonucleotides have the following sequences and modifications:

sequencing primer
(SEQ ID NO: 40)
5′-GGGACTCGAGTGATGATTGG-3′
5′-phosphorylated amplification primer
(SEQ ID NO: 41)
/5Phos/ /5Phos/CCACATTACATACTTACCATGCC-3′
Blocking oligo (BNA)
(SEQ ID NO: 42)
5′-ACCATGCCACTTTCCCTTGTAGACTGTT+C+CAAATGAT+CCAGA
T+CCAATTC/3Phos/;

where /5Phos/ stands for 5′-phosphorylation, “+” for locked nucleic acid (LNA), and /3Phos// for 3′-phosphorylation.

Stock plasmids (clones of BRAF) and dilutions thereof were used (10,000 copies/μL) in the experiments described herein. The nucleic acid samples were prepared using standard protocols, amplified by PCR and sequenced in a reaction mixture containing 2.5 μL Better Buffer (The Gel Company), 0.25 μL Big Dye v.3.1 (Applied Biosystems), 0.13 μL 10 mM dNTPs, 1 μL 10 μM sequencing primer, 1 μL 1 μM 5′-phosphorylated amplification primer, 1.6 μL (or optimized) 2.5 μM Blocking nucleic acid and 1 μL DNA template to a total volume of 10 μL per reaction. The reaction was carried out in a thermal cycler as follows: 40 cycles of 95° C. for 15 sec, 70° C. for 2 minutes, the critical temperature for 30 seconds, 50° C. for 10 seconds and 60° C. for 45 seconds followed by incubation at 12° C. The lambda exonuclease (0.5 μL at 5,000 U/mL) was then added to the reaction mixture and incubated at 37° C. for 30 minutes to degrade the amplified strand comprising the 5′-phosphate. The critical temperature for V600E for sequencing is 77.6° C. and for ICE COLD PCR is 76.4° C. The critical temperature for G469A for sequencing is 74.6° C. and for ICE COLD PCR is 73.2° C. Finally, the material is further purified as for standard sequencing according to the CleanSEQ protocol (Agencourt Biosciences). The Tcs were determined and the concentrations of the BNA used were optimized as described above.

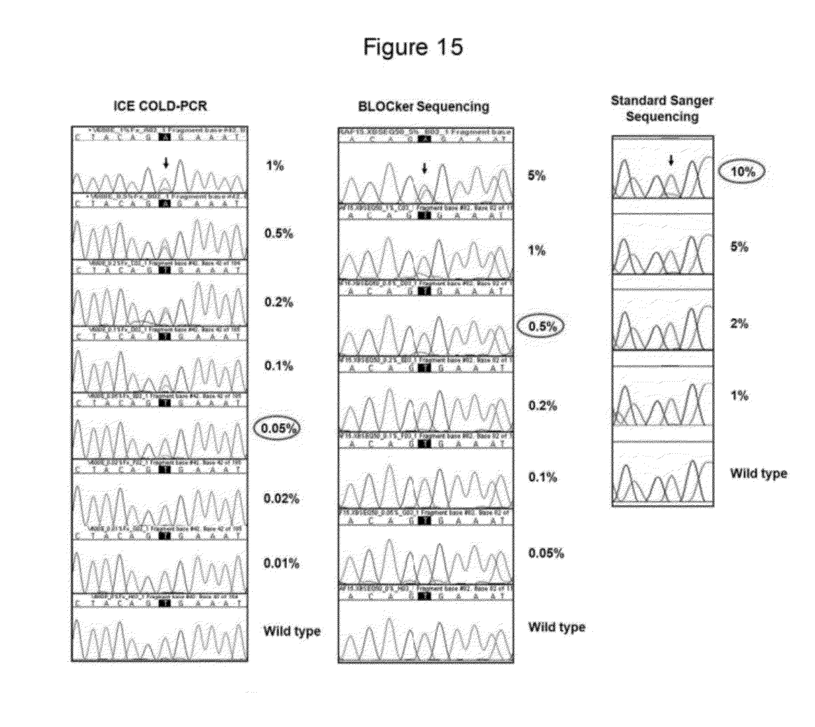

FIG. 15 shows the electropherograms for detection of the V600E BRAF exon 15 mutation in the background of an excess of wild-type sequence (SEQ ID NO:43; 5′-CTACAGA/TGAAAT-3′; the underlined bases are the site of mutation with the first base being the mutant and the one after the slash the wild-type). The percentages indicate the percentage of mutant target in the total DNA template added to the reaction mixture. The first electropherogram demonstrates that the limit of detection of the target V600E mutation is 0.05% by ICE COLD PCR, the middle electropherogram shows the reaction described herein provides a limit of detection of 0.5% and standard sequencing, shown in the electropherogram on the right shows that standard sequencing provides a limit of detection of 10%.

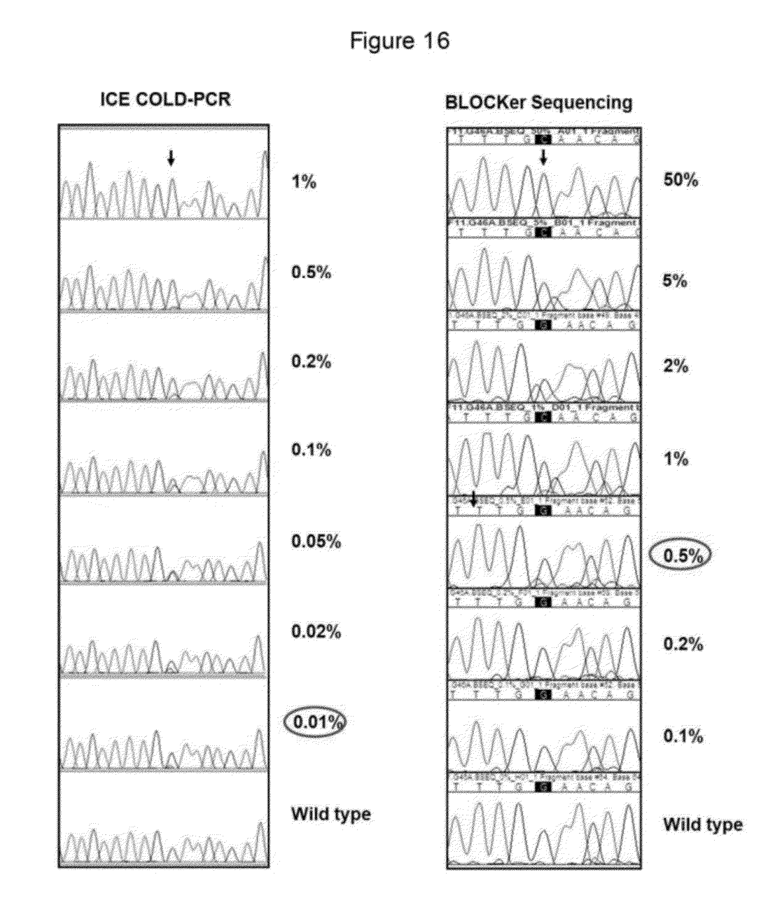

FIG. 16 shows the electropherograms for detection of the G469A BRAF exon 11 mutation in the background of an excess Of wild-type sequence (SEQ ID NO:44; 5′-TTTGC/GAACAG-3′; the underlined bases are the site of mutation with the first base being the mutant and the one after the slash the wild-type). The percentages indicate the percentage of mutant target in the total DNA template added to the reaction mixture. The left electropherogram demonstrates that the limit of detection of the target G469A imitation is 0.01% by ICE COLD PCR. The electropherogram on the right shows the BLOCker sequencing reaction described herein provides a limit of detection of 0.5%. We expect that a combination of ICE COLD PCR and the BLOCker sequencing reaction, instead of traditional PCR and BLOCker sequencing as described herein, would result in a still lower limit of detection.

We claim:

1. A kit for sequencing a target DNA sequence in a sample having a reference sequence comprising a sequencing primer and a blocking nucleic acid, the sequencing primer is complementary to a portion of one strand of the target sequence and the reference sequence, the blocking nucleic acid is fully complementary with at least a portion of one strand of the reference sequence, wherein the sequencing primer and the blocking nucleic acid are complementary to the same strand of the reference sequence, and wherein the blocking nucleic acid is blocked at the 3′ end such that it cannot be extended by a polymerase.

2. The kit of claim 1, further comprising labeled chain terminating nucleotide triphosphates.

3. The kit of claim 1, wherein the target sequence and the reference sequence can be denatured to produce target strands and reference strands, and wherein the blocking nucleic acid is capable of forming a homoduplex with the fully complementary reference strand and a heteroduplex with the partially complementary target strand when allowed to hybridize.

4. The kit of claim 3, wherein heteroduplexes of the blocking nucleic acid and the complementary target strand denature at a lower temperature than duplexes of the blocking nucleic acid and the complementary reference strand.

5. The kit of claim 4, wherein the sequencing primer is capable of annealing to the target strand at a temperature below the critical temperature.

6. The kit of claim 1, wherein the 3′ end of the sequencing primer is capable of binding to a strand of the reference sequence near to the base on the strand of the reference sequence that binds the 5′ end of the blocking nucleic acid or the 3′ end of the sequencing primer is complementary to at least one of the same bases of the reference sequence as the 5′ end of the blocking nucleic acid.

7. The kit of claim 1, wherein a 5′-end on the blocking nucleic acid comprises a nucleotide that prevents 5′ to 3′ exonucleolysis by DNA polymerases.

8. The kit of claim 1, wherein the blocking nucleic acid is a single-stranded nucleic acid.

9. The kit of claim 1, wherein the blocking nucleic acid comprises DNA, RNA, peptide nucleic acid, locked nucleic acid, another modified nucleotide or a combination thereof.

10. The kit of claim 9, wherein the position of a peptide nucleic acid, locked nucleic acid or another modified nucleotide in the blocking nucleic acid is selected to match a position where the reference sequence and the target sequence are suspected to be different.

11. The kit of claim 10, whereby the difference between the temperature needed to denature heteroduplexes of the blocking nucleic acid and a complementary target strand and the temperature needed to denature duplexes of the blocking nucleic acid and a complementary reference strand is maximized.

12. The kit of claim 9, wherein the position of a peptide nucleic acid, locked nucleic acid or another modified nucleotide in the blocking nucleic acid is selected to provide a more constant melting temperature across the blocking nucleic acid.

13. The kit of claim 1, further comprising a 5′-phosphorylated primer, wherein the 5′-phosphorylated primer is not complementary to the same strand as the sequencing primer.

14. The kit of claim 13, further comprising a 5′-phosphate dependent exonuclease.

15. The kit of claim 1, wherein the target sequence or the reference sequence comprises K-RAS exon 2 codon 12 and/or 13.

16. The kit of claim 1, wherein the target sequence or the reference sequence comprises a mitochondrial imitation.

17. The kit of claim 16, wherein the mitochondrial mutation is associated with MELAS.

18. The kit of claim 1, wherein the target sequence or the reference sequence comprises HPV nucleic acid.

19. The kit of claim 1, wherein the target sequence or the reference sequence comprises BRAF exon 11 and/or exon 15.

20. A method for preparing a target sequence in a sample for sequencing comprising:
a) adding the sample to a DNA sequencing reaction mixture to form a reaction mixture,
the sample having a reference sequence and also suspected of having one or more target sequences and the DNA sequencing reaction mixture comprising a sequencing primer and a molar excess amount of a blocking nucleic acid that is fully complementary with at least a portion of one strand of the reference sequence,
wherein the blocking nucleic acid and the sequencing primer are complementary to the same strand of the reference sequence, and
wherein the blocking nucleic acid is blocked at the 3′ end such that it cannot be extended by a polymerase;
b) subjecting the reaction mixture suspected of having the target sequence to a first denaturing temperature that is above the melting temperature (Tₘ) of the reference sequence and the target sequence to form denatured reference strands and denatured target strands;
c) reducing the temperature of the reaction mixture to permit formation of duplexes of the blocking nucleic acid and the complementary reference strand and heteroduplexes of the blocking sequence and target strands;
d) increasing the temperature of the reaction mixture to a critical temperature (T_{c}) sufficient to permit denaturation of said heteroduplexes of the blocking nucleic acid and the complementary target strands, yet insufficient to denature duplexes of the blocking nucleic acid and the complementary reference strand;
e) reducing the temperature of the reaction mixture to permit the sequencing primer to anneal to free target strands and free reference strands in the reaction mixture; and
f) extending the sequencing primer to generate extension products, the extension products capable of being analyzed to allow determination of the nucleic acid sequence of the target sequence.

21. The method of claim 20, further comprising determining the nucleic acid sequence of the target sequence.

22. The method of claim 21, wherein the sequence is determined by di-deoxy-sequencing, single-molecule sequencing, pyrosequencing, or second generation high-throughput sequencing.

23. The method of claim 20, wherein the 3′ end of the sequencing primer binds to a strand of the reference sequence near to the base on the reference strand that binds the 5′ end of the blocking nucleic acid or the 3′ end of the sequencing primer is complementary to at least one of the same bases of the reference sequence as the 5′ end of the blocking nucleic acid.

24. The method of claim 20, wherein the 3′ end of the sequencing primer and the 5′ end of the blocking nucleic acid are complementary to more than one of the same bases of a strand of the reference sequence.

25. The method of claim 20, wherein a 5′ end on the blocking nucleic acid comprises a nucleotide that prevents 5′ to 3′ exonucleolysis by DNA polymerases.

26. The method of claim 20, wherein the blocking nucleic acid comprises DNA, RNA, peptide nucleic acid, locked nucleic acid, another modified nucleotide or a combination thereof.

27. The method of claim 26, wherein the position of a peptide nucleic acid, locked nucleic acid or another modified nucleotide in the blocking nucleic acid is selected to match a position where the reference sequence and the target sequence are suspected to be different.

28. The method of claim 26, wherein the position of a peptide nucleic acid, locked nucleic acid or another modified nucleotide in the blocking nucleic acid is selected to provide a more constant melting temperature across the blocking nucleic acid.

29. The method of claim 20, wherein the sequencing primer is capable of annealing to a strand of the reference sequence at a temperature below the critical temperature.

30. The method of claim 20, wherein the sequencing primer is added to the reaction mixture in molar excess to the blocking nucleic acid.

31. The method of claim 20, wherein the melting temperature of duplexes of the reference strand and blocking nucleic acid is higher than the melting temperature of heteroduplexes of the target strand and blocking nucleic acid.

32. The method of claim 20, further comprising amplifying at least one of the target sequences in the sample prior to using at least a portion of the amplification product as the sample in step (a) by including an amplification primer in the reaction mixture.

33. The method of claim 32, further comprising selectively degrading one strand of the amplified product.

34. The method of claim 33, wherein the amplification primer is labeled to allow for the resulting labeled target strand to be degraded.

35. The method of claim 34, wherein the amplification primer is labeled with a 5′-phosphate and the method further comprises incubating the sequencing reaction with a 5′-phosphate dependent exonuclease.

36. The method of claim 20, wherein said method is repeated for two or more cycles in a cycle sequencing reaction.

37. The method of claim 20, wherein said reaction mixture contains a nucleic acid detection dye.
